(19) United States
(12) Patent Application Publication
(10) Pub. No.: US 2005/0031703 A1
(43) Pub. Date: Feb. 10, 2005

(54) Antimicrobial, water-insoluble silicate glass powder and mixture of glass powders

(75) Inventors: Wolfram Beier, Essenheim (DE); Jorg Hinrich Fechner, Mainz (DE); Rupert Schnell, Worms (DE); Jose Zimmer, Ingelheim (DE)

Correspondence Address: CHARLES N.J. RUGGIERO, ESQ.;OHLANDT, GREELEY, RUGGIERO & PERLE, L.L.P., ONE LANDMARK SQUARE, 10th FLOOR, STAMFORD, CT, 06901-2682, US

(73) Assignee: Schott Glas

(21) Appl. No.: 10/897,689

(22) Filed: Jul. 23, 2004

Related U.S. Application Data
(63) Continuation of application No. PCT/EP03/00559, filed on Jan. 21, 2003.

(30) Foreign Application Priority Data
Jan. 24, 2002 (DE) 102 02 630.0

(51) Int. Cl. A61K 33/42; C03C 3/118; C03C 3/064; A61K 33/38

(52) U.S. Cl. 424/601; 501/77; 424/618

(57) ABSTRACT

An antimicrobial, water-insoluble silicate glass powder is provided. The starting glass includes 30 to 70 weight percent SiO₂, 0 to 1 weight percent Na₂O, 0 to 1 weight percent K₂O, 0 to 40 weight percent MgO, 0 to 40 weight percent CaO, 0 to 40 weight percent SrO, 0 to 40 weight percent BaO, 0 to 25 weight percent Al₂O₃, 0 to 20 weight percent P₂O₅, 0 to 20 weight percent B₂O₃, wherein the sum of the alkali oxide contents is less than 1.5 weight percent in the total composition of the starting glass. The starting glass also includes, as biocidally active components, ions or atoms of the elements Ag, Zn, Cu, Ce, Te, or I with total proportions of greater than 2.5 weight percent.

### BACKGROUND OF THE INVENTION

The invention concerns antimicrobial, water-insoluble silicate glass powders and mixtures made up of glass powders. Antimicrobial glass powders are understood to be glass powders that, for example, have a biocidal, bacteriocidal, and/or fungicidal and/or algicidal effect. In this application, glass powders are understood generally to be glasses in powder form or fiber form or in the form of fine particles.

1. Field of the Invention

Glasses with a bioactive effect and also partially an antimicrobial effect are described as bioglass by L. L. Hensch, J. Wilson, An Introduction to Bioceramics, World Scientific Publ. 1993. Such bioglass is characterized by the formation of hydroxyapatite layers in aqueous media.

2. Description of Related Art

For the glasses and glass powders known from prior art, the antibacterial effect is due to the added heavy metal ions, such as, for example, Cu, Zn, or Ag. The antibacterial effect for these compounds does not arise from glass, but rather from the metal ions released.

Thus, in U.S. Pat. No. 5,290,544, water-soluble glasses with very low Sio₂ and very high B₂O₃ or high P₂O₅ contents are described for application in cosmetic products. The glasses have silver concentrations greater than 0.5 wt.%. These glasses have an extremely low hydrolytic resistance and tend to dissolve completely in water. The Ag and/or Cu ions hereby released have an antibacterial effect. Described in JP A 92 178,433 is also a water-soluble glass powder with SiO₂<37 wt.% as a polymer additive with a high silver concentration of >1 wt.%.

Described in U.S. Pat. No. 6,143,318 are silver-containing phosphate glasses that are used as antimicrobial material for wound-infection treatment with combinations of Cu, Ag, and Zn. Involved here are also water-soluble glasses that have low SiO₂ concentrations and very high P₂O₅ contents.

Glasses with a high proportion of phosphorus that are largely free of alkali components are described in EP 1,116,698 and in JP 08[1996]175,843.

### BRIEF DESCRIPTION OF THE INVENTION

Owing to their low hydrolytic resistance, these glasses are of only very limited suitability for pulverizing in aqueous media.

Involved in the case of the glasses known from WO 01/03650 are bioactive glasses with a significant phosphorus proportion of >1 wt.%.

### DETAILED DESCRIPTION OF THE INVENTION

The key properties of bioactive glass are known to the person skilled in the art and are described in, for example, U.S. Pat. No. 5,074,916. In accordance therewith, bioactive glass differs from conventional soda-lime-silicate glasses in that it binds living tissue.

Bioactive glass refers to a glass that binds strongly to body tissue with the formation of a hydroxyapatite layer.

A drawback of these bioactive glasses is, in turn, the high proportion of phosphorus, which, when the molten glasses are prepared, leads to fabrication problems and to a low hydrolytic resistance.

A further drawback of the glasses described in the prior art is that these substances have adverse side effects and are not unobjectionable in terms of health. They can cause allergies, irritate the skin, or, in another form, be detrimental to the human body or the environment.

In particular, when they are used in certain polymers comprising plastics or lacquers, powders made up of such glasses have the drawback that the polymer chain is cleaved and the polymeric material is thereby locally destroyed. The mechanical and optical properties of the polymeric material are hereby permanently impaired.

The object of the invention is to provide glass powders that avoid the disadvantages of the prior art and, in particular, can be prepared without great effort or expense.

This object is solved by a glass powder according to claim 1.

The glass of the invention can be produced on a large industrial scale by use of standard processes.

Owing to their hydrolytic resistance, the starting glasses can be pulverized in different pulverizing media, such as, for example, in water.

The antimicrobial glasses allow the products themselves to be preserved or an outwardly directed antimicrobial effect is achieved.

A particular advantage of the glass of the invention is that, owing to its melting and hot-forming behavior, the glass is suitable for production in corresponding large-scale industrial plants.

A further advantage is that such silicate glass powders can be incorporated as admixtures into polymers comprising plastics or lacquers, without the polymer chains being cleaved. For example, the polymer chains in polycarbonates, in particular, are also readily attacked, so that the mechanical and optical properties of polycarbonates are not influenced detrimentally by the silicate glass powders of the invention as admixtures.

Because of the low process temperatures or the viscosity of the glass, it is possible to employ cost-effective materials for melts and hot-forming.

Besides the production by way of a melt process, alternative production processes by way of the sol-gel or reaction sintering route are also conceivable.

The antimicrobial effect of the glass powder of the invention is extremely strong. The smaller the mean particle size of the glass powder, the higher the antimicrobial effect because of the increase in the reactive surface of the glass. The antimicrobial properties are also found for glasses that, as semifinished products, have a relatively high hydrolytic resistance.

For the glasses of the invention, alkaline earths of the glass are replaced by H⁺ ions of the aqueous medium by reactions on the surface of the glass. The antimicrobial effect of the ion exchange is due to, among other things, an increase in the pH and to the osmotic effect on microorganisms.

Ion-exchanging glasses according to the invention act antimicrobially in aqueous media owing to an increase in the pH due to ion exchange between a metal ion, such as, for example, an alkaline-earth metal ion, and the H⁺ ions of the aqueous solution as well as due to impairment of cell growth (osmotic pressure, disruption of cellular metabolic processes) caused by ions. Pulverized glass powders with particles of small particle size and large surface show a dramatic increase in reactivity, from which, by means of the ion exchange already described, a strong antimicrobial effect results. The antimicrobial effect for the glass powders of the invention is achieved by means of the ion exchange and not by the antimicrobial effect of the heavy metal ions. However, the latter, as an additive, can augment the antimicrobial effect.

A pulverizing process can yield particle sizes of <100 μm. Particle sizes of <50 μm or 20 μm have proven appropriate. Particularly suitable are particle sizes of <10 μm as well as smaller than 5 μm. Particle sizes of <1 μm have been found to be quite especially suitable.

The pulverizing process can be carried out both in a dry manner and with aqueous and nonaqueous pulverizing media.

Depending on the particle size, the concentration, and the composition of the powder, pH values of up to 13 are reached.

The glass contains SiO₂ as a network former, preferably between 35 and 80 wt.%. At low concentrations, the hydrolytic resistance decreases strongly, so that pulverizing in aqueous media is no longer ensured without significant dissolution of the glass.

Alkaline-earth oxides can be added, in particular, in order to increase the ion exchange and thus to augment the antimicrobial effect. Al₂O₃ can be added in an amount of up to at most 25 wt.% in order to increase the chemical resistance of the crystallization stability and the control of the antimicrobial effect.

B₂O₃ acts as a network former and can serve as well to control the antimicrobial effect.

Ag₂O, CuO, ZnO can be added as antimicrobially acting additives, which augment the intrinsic antimicrobial effect of the base glass in a synergistic manner.

Through a combination of the pH effect and the release of Ag, Cu, or Zn, it is possible to achieve a substantial increase in the antimicrobial effect, which markedly exceeds the sum of the individual effects. The concentrations of Ag, Cu, Zn ions hereby released into the product can lie clearly below 1 ppm.

The incorporation of Ag, Cu, Zn can hereby take place already in the melt by way of corresponding salts or else through ion exchange of the glass after the melting.

Owing to their biocidal, bacteriocidal, and fungicidal effects, antimicrobial glass powders are suitable as admixtures or fillers for the most diverse purposes. However, problems arise during application, particularly in regard to the intensity of the effect over time.

In accordance with the invention, this aspect of the invention is solved by making available mixtures of glass powders, which comprise glass powders with different time-release properties of the active components.

Such mixtures can be, for example, binary, tertiary, or quaternary mixtures of different fractions of glass powders. It is particularly preferred when the mixture involves a binary mixture made up of two fractions of glass powders with different time-release properties.

In order to be able to employ such mixtures also in plastic products or products that contain polymeric materials, it is preferred to employ glass powders that are totally alkali-free or are alkali-poor and referred to as “practically alkali-free.” Preferably, the total content of sodium and/or potassium oxide, for example, is less than 1.5 wt.%.

The idea of the mixture of glass powders with different release rates for achieving a biocidal effect that is as uniform as possible is not limited to alkali-free glasses, however. Thus, in other fields of application, alkali-free glasses may not be necessary.

One of the glass powders in a binary mixture comprises a first fraction of a component with low release rate and, accordingly, a continuous antimicrobial efficacy and the other glass component of the second fraction comprises components with a rapid release rate and, accordingly, with a short-term antimicrobial effect.

Glass powders of alkaline-earth borate glasses, alkaline-earth phosphate glasses, or zinc phosphate glasses have proven to be particularly preferred for an immediate biocidal effect.

The antimicrobial or biocidal property in the glass powders is achieved through ions or atoms of the elements Ag, Zn, Cu, Ce, Te, or I with total proportions of <2.5 wt.%. The biocidal effect occurs, for example, against bacilli, fungi, algae, and other microorganisms.

Preferably, one or more biocidally active components, selected from the following components: Ag₂O, CuO, Cu₂O, TeO₂, ZnO, CeO₂, and I, are added to the base glass of the glass powder.

Glass powders with slow release and thus long-term effect are, above all, glass powders of silicate glasses. These, too, can comprise the previously mentioned components, which augment the antimicrobial effect. Preferably, the glass powders in both cases are free or nearly free of alkali components.

If a silicate glass powder is used for slow release, then, preferably, a silicate glass with the following components is used as the starting glass:

| | |
|---|---|
| SiO₂ | 30-70 wt. % |
| Na₂O | 0-1 wt. % |
| K₂O | 0-1 wt. % |
| MgO | 5-40 wt. % |
| CaO | 0-40 wt. % |
| SrO | 0-40 wt. % |
| BaO | 0-40 wt. % |
| Al₂O₃ | 0-25 wt. % |
| P₂O₅ | 0-20 wt. % |
| B₂O₃ | 0-20 wt. % |

The antimicrobial or biocidal property in the glass powders is achieved through ions or atoms of the elements Ag, Zn, Cu, Ce, Te, or I with total proportions of <2.5 wt.%. The biocidal effect occurs, for example, against bacilli, fungi, algae, and other microorganisms.

Ag₂O, CuO, Cu₂O, TeO₂, ZnO, CeO₂, and I can be added as biocidally active components to the base glass of the glass powder.

For both fractions of glass powders, it may be advantageous to allow the fraction with the higher release rate of the biocidal components to be active particularly at elevated humidity.

The particle size of the various glass powders can differ. Thus, the mean particle size of a glass powder with particle size distribution is smaller than 100 μm, preferably smaller than 30 μm, particularly preferably smaller than 5 μm, and most preferably smaller than 1 μm.

It may be appropriate to use a glass powder mixture made up of two fractions, wherein the mean particle sizes clearly differ from each other on account of the different particle size distributions of the two fractions, particularly in such a manner that the fraction with the lower release rate, that is, the first release rate, of the biocidal components consists of particles that are smaller by a factor of 2 to 5 than the particles of the glass powder with the higher release rate, that is, the second release rate, of the biocidal components.

In a further embodiment, it can be provided that the particle size distributions of the two fractions markedly differ from each other, particularly in such a manner that the fraction with the lower release rate of the biocidal components consists of particles that are smaller by a factor of 5 to 20 than the particles of the glass powder with the higher release rate of the biocidal components.

Further, a particle dimensioning can be conducted in such a way that the fraction with the lower release rate of the biocidal components consists of particles that are larger by a factor of 2 to 5 than the particles of the glass with the larger release rate of the biocidal components.

It is also conceivable that the particle size distributions of the two fractions clearly differ from each other, particularly in such a manner that the fraction with the lower release rate of the biocidal components consists of particles that are larger by a factor of 5 to 20 than the particles of the glass with the larger release rate of the biocidal components.

The antimicrobial glass powders, particularly the mixtures with biocidal effect in accordance with the invention come into consideration for the following application objectives, particularly as admixtures:
for plastics in exterior applications, such as, for example, carport roofs;
for insoles and inlay soles in shoes to prevent odor or act against athlete's foot;
for hygienic applications;
for medical applications, such as catheters, cannulas;
for paints and plastics for underwater applications;
for industrial applications, such as, for example, cooling system circuits with plastic
piping in order to suppress the growth of algae, which is often disruptive;
for plastics based on polyester;
for trim, such as, for example, plastic frames of refrigerator shelves;
sensors for household appliances;
switch devices, such as, for example, pushbuttons for household appliances;
trim, such as, for example, seals of glass surfaces of beverage vending machines;
for disinfection devices in the kitchen;
for trim, such as, for example, seals of the front doors of baking ovens:
for microwave devices;
for ventilation hoods;
for trim, such as, for example, plastic frames of glass ceramic cooking surfaces;
for trim of glass surfaces in saunas, solaria, showers;
for fitness equipment;
for devices in medical technology, such as patient scales, etc.;
for surfaces of display devices, such as displays, touchscreens, etc.;
for trim of furniture glass;
for plastic parts of game machines;
for trim, particularly seals of construction glass, particularly interior doors.

Particularly favorable effects have been found in the application for plastics. Thus, for example, the mechanical and optical properties of polycarbonates remain largely unimpaired. The ester bond of the polymer chain hereby remains untouched and is not cleaved.

The mixtures of the invention are suitable particularly as additives for the production of antimicrobial surfaces, such as, for example, trim of glass or glass ceramic parts. The trim can involve, for example, seals or plastic frame parts. In particular, the glass finds use as an admixture to plastics that are used for the production of plastic frames for refrigerator shelves. The glass can also be employed as an admixture to plastics that are used for plastic switches, for example, on stoves.

The invention will be described in greater detail below on the basis of embodiment examples:

### EMBODIMENT EXAMPLES

The molten glass was prepared from the raw materials in a platinum crucible at 1600° C. and processed into semifinished product or ribbons, that is, glass strips. The ribbons were pulverized in a drum mill to particle sizes of up to 4 μm. Particle sizes of less than 4 μm were attained by attritor pulverizing in aqueous or nonaqueous medium. The compositions of the starting glasses of the invention from which glass powders were produced by pulverizing are presented in the following Table 1.

TABLE 1
Compositions of starting glasses of the invention:

| | Embodiment | Embodiment |
|---|---|---|
| | Example 1 | Example 2 |
| SiO₂ | 56.0 | 59.85 |
| Al₂O₃ | 15.8 | 16.50 |
| CaO | 8.4 | 13.50 |
| MgO | 5.6 | 0.0 |
| Fe₂O₃ | | |
| B₂O₃ | 4.8 | 0.3 |
| SrO | | |
| BaO | 8.4 | 7.85 |
| ZrO₂ | | 1.0 |
| Ag₂O | 1.0 | 1.0 |

The compositions given are to be understood as being by way of example for glasses with an alkali oxide content of less than 1.5 wt.% and are in no way limited to the special embodiment examples given.

1-19. (Cancelled)

20. An antimicrobial, water-insoluble silicate glass powder, comprising:
30 to 70 weight percent SiO₂;
0 to 1 weight percent Na₂O;
0 to 1 weight percent K₂O;
0 to 40 weight percent MgO;
0 to 40 weight percent CaO;
0 to 40 weight percent SrO;
0 to 40 weight percent BaO;
0 to 25 weight percent Al₂O₃;
0 to 20 weight percent P₂O₅;
0 to 20 weight percent B₂O₃; and
a biocidally active component, said biocidally active component having ions or atoms of an element selected from the group consisting of Ag, Zn, Cu, Ce, Te, I, and any combinations thereof, said biocidally active component having a total proportion of less than 2.5 weight percent, wherein a sum of alkali oxide contents is less than 1.5 weight percent, and wherein the silicate glass powder has a particle size of less than 100 μm.

21. The antimicrobial, water-insoluble silicate glass powder according to claim 20, wherein said MgO is present in at least 5 weight percent.

22. The antimicrobial, water-insoluble silicate glass according to claim 20, wherein said element is selected from the group consisting of Ag₂O, CuO, Cu₂O, TeO₂, ZnO, CeO₂, I, and any combinations thereof, and wherein said total proportion is greater than 0.1 weight percent.

23. The antimicrobial, water-insoluble silicate glass powder according to claims 20, wherein said particle size is less than or equal to 20 μm.

24. The antimicrobial, water-insoluble silicate glass powder according to claims 20, wherein said particle size is less than or equal to 10 μm.

25. The antimicrobial, water-insoluble silicate glass powder according claims 20, wherein said particle size is less than 5 μm.

26. The antimicrobial, water-insoluble silicate glass powder according to claim 20, wherein said particle size is less than 1 μm.

27. The antimicrobial, water-insoluble silicate glass powder according to claim 20, wherein the silicate glass powder is usable as a functional additive or filler for a plastic selected from the group consisting of thermoplastic, duroplastic, and elastomeric plastic.

28. The antimicrobial, water-insoluble silicate glass powder according to claim 27, wherein said plastic is a polycarbonate based plastic or polyester based plastic.

29. The antimicrobial, water-insoluble silicate glass powder according to claim 20, wherein the silicate glass powder is usable in polymer-containing dyes and paints.

30. A plastic product having a biocidal property, comprising:
a plastic selected from the group consisting of thermoplastic, duroplastic, and elastomeric plastic; and
a water-insoluble silicate glass powder having
30 to 70 weight percent SiO₂;
0 to 1 weight percent Na₂O;
0 to 1 weight percent K₂O;
0 to 40 weight percent MgO;
0 to 40 weight percent CaO;
0 to 40 weight percent SrO;
0 to 40 weight percent BaO;
0 to 25 weight percent Al₂O₃;
0 to 20 weight percent P₂O₅;
0 to 20 weight percent B₂O₃; and
a biocidally active component, said biocidally active component having ions or atoms of an element selected from the group consisting of Ag, Zn, Cu, Ce, Te, I, and any combinations thereof, said biocidally active component having a total proportion of less than 2.5 weight percent of said water-insoluble silicate glass powder, wherein a sum of alkali oxide contents is less than 1.5 weight percent of said water-insoluble silicate glass powder, and wherein said water-insoluble silicate glass powder has a particle size of less than 100 μm.

31. The plastic product according to claim 30, wherein said water-insoluble silicate glass powder is exclusively or principally located at a surface of the plastic product.
